(19) United States
(12) Patent Application Publication
(10) Pub. No.: US 2013/0340771 A1
(43) Pub. Date: Dec. 26, 2013

(54) Post Operative Amputated Lower Leg Limb Protective and Promotive Healing Device

(71) Applicant: Samuel L. Morris, III, Buffalo, NY (US)

(72) Inventor: Samuel L. Morris, III, Buffalo, NY (US)

(73) Assignee: MILSPORT MEDICAL PRODUCTS, LLC, Buffalo, NY (US)

(21) Appl. No.: 13/533,239

(22) Filed: Jun. 26, 2012

(51) Int. Cl. A61F 5/40; B82Y 5/00

(52) U.S. Cl. 128/888; 977/773; 977/931

(57) ABSTRACT

A post operative amputated lower leg limb protective and promotive healing device including a supportive section having a first end and an opposite second end; and an open framework protective section releasably securable to at least one end of the supportive section. The open framework protective section forms a plurality of openings to allow airflow therethrough in all directions and is configured to insure no physical contact by the protective section with the amputated lower leg limb. The open framework protective section is non-weight bearing.

### BACKGROUND OF THE INVENTION

1. Field of the Invention

The present invention is directed to a post operative lower limb protective and healing promotion device. In particular, the invention relates to a post operative device that serves the dual purpose of providing protection to a lower leg limb following surgical amputation while simultaneously promoting healing.

2. Description of Related Art

Amputation of a lower limb either transtibial (below the knee) or transfemoral (above the knee) is a surgical operation in which following the procedure the stump or remaining portion of the extremity that has been amputated is sore and tender. Coinciding with this same period the patient is often mobile using a wheelchair with the leg on which the surgical procedure has been performed resting on an elevated leg rest in which the stump is vulnerable to blunt traumatic impact with an object. Accordingly, it would be desirable to develop a protective device that minimizes the chance of impact with the stump.

Moreover, amputation of a lower limb is a procedure commonly associated with patients exhibiting Type 2 diabetes due to poor circulation and or neuropathy. Thus, healing is a primary concern for all amputees, but has even greater significance when the patient is a diabetic for whom the healing of any wound may be of significant concern and more challenging. Healing is promoted by the free circulation or ventilation of air surrounding the area of the wound with minimal, if any, physical impediment, pressure or contact with the wound.

It is therefore desirable to develop a post operative lower limb protective and healing promotion device that solves the aforementioned problems associated with conventional devices.

### SUMMARY OF THE INVENTION

The present invention is directed to a post operative lower limb protective and healing promotion device that solves the aforementioned problems associated with conventional devices.

Another aspect of the present invention is directed to a post operative amputated lower leg limb protective and promotive healing device that suspends the stump so as not to be in physical contact with any other object.

Yet another aspect of the present invention is directed to a post operative amputated lower leg limb protective and promotive healing device including a supportive section having a first end and an opposite second end; and an open framework protective section releasably securable to at least one end of the supportive section. The open framework protective section forming a plurality of openings to allow airflow therethrough in all directions; and the open framework protection section being configured to insure no physical contact by the protection section with the amputated lower leg limb; wherein the open framework protective section is non-weight bearing.

Still another aspect of the present invention is directed to a method for using the post operative amputated lower leg limb protective and promotive healing device as described in the preceding paragraph. The method includes the step of placing the amputated lower leg limb into the supportive section until at least a portion of an end of the amputated lower leg limb extends beyond the second end of the supportive section, that portion of the amputated lower leg limb extending beyond the second end of the supportive section remaining free from any support or physical contact whatsoever with the device.

### BRIEF DESCRIPTION OF THE DRAWING

The foregoing and other features of the present invention will be more readily apparent from the following detailed description and drawings of illustrative embodiments of the invention wherein like reference numbers refer to similar elements throughout the several views and in which:

FIG. 1 is a perspective view of an exemplary post operative lower limb protective and healing device in accordance with the present invention;

FIG. 2 is a top view of the post operative lower limb protective and healing promotion device of FIG. 1;

FIG. 3 is a bottom view of the post operative device of FIG. 1;

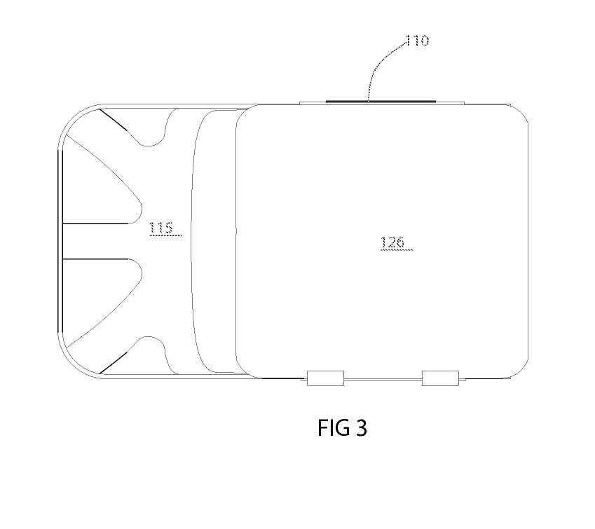

FIG. 4A is a right side view of the post operative device of FIG. 1 with the straps in a released, extended state;

FIG. 4B is a right side view of the post operative device of FIG. 1 with the straps in a secured state;

FIG. 5 is a left side view of the post operative device of FIG. 1 with the straps in a secured state;

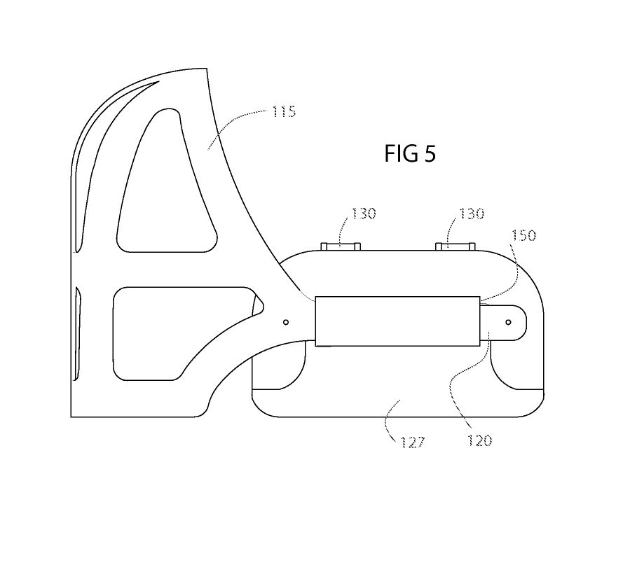

FIG. 6 is a back view of the post operative device of FIG. 1 when viewed from the supporting section;

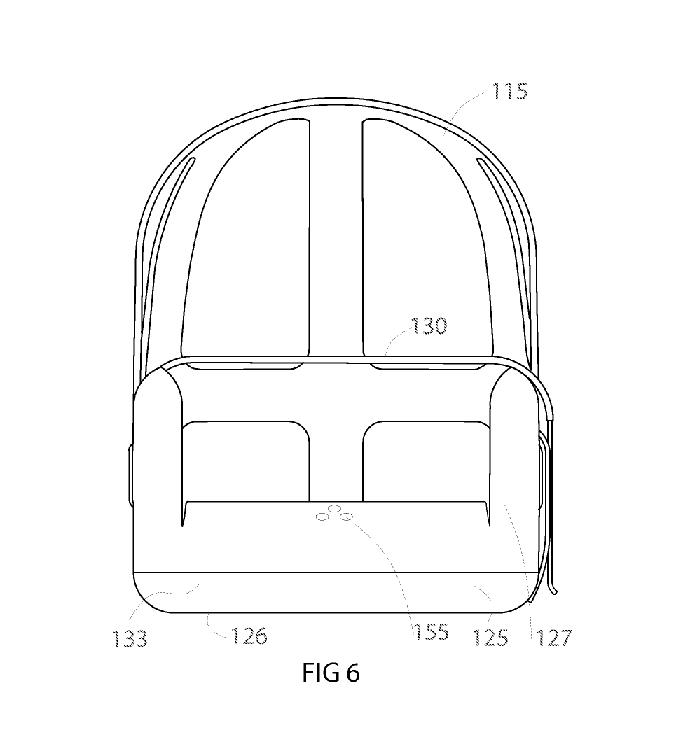

FIG. 7 is a front view of the post operative device of FIG. 1 when view from the protective section;

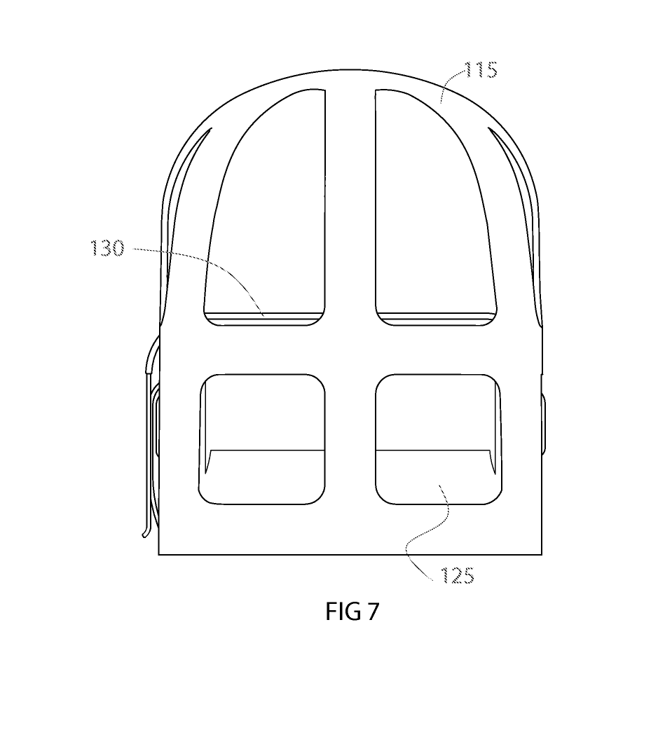

FIG. 8A is a perspective view of the post operative device of FIG. 1 being worn by a patient having a right leg transtibial amputation;

FIG. 8B is a perspective view of the post operative device of FIG. 1 being worn by a patient having a right leg transfemoral amputation;

FIG. 8C is a longitudinal cross-sectional view of the post operative device of FIG. 8A;

FIG. 8D is a longitudinal cross-sectional view of the post operative device of FIG. 8B; and

FIG. 9 is a cross-sectional view of the supportive section along lines IX-IX in FIG. 2 without the straps.

### DETAILED DESCRIPTION OF THE PRESENTLY PREFERRED EMBODIMENTS OF THE INVENTION

The present invention is directed to a post operative protective and healing promotive device to be used following amputation surgery to remove a lower leg limb. A single device may be universally used with either transtibial (i.e., below the knee) or transfemoral (i.e., above the knee) amputation, as well as be interchangeably employed on either the left or right hand side of the body. To accommodate individuals of varying size, the device is preferably manufactured in a series of two or more different sizes, for example, four different sizes (small, medium, large, extra large). The sizes differ from one another in overall dimensions of the protective and supportive sections of the device.

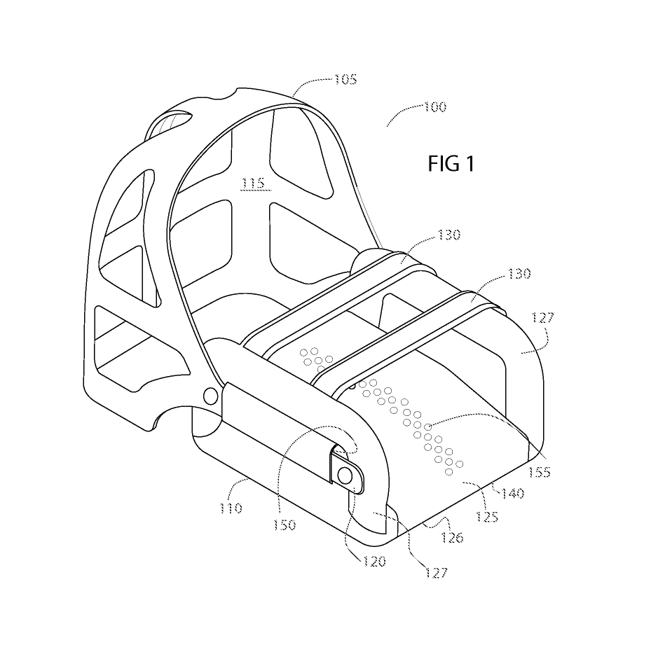

FIG. 1 is a perspective view of an exemplary embodiment of the post operative lower leg limb protective and healing promotion device 100 having two sections, e.g., a protective section 105 and a supportive section 110 releasably securable to one another. Protective section 105 protects the stump suspended therein against blunt trauma and direct impact with an object without physically contacting the stump thereby promoting air circulation and healing. Suspended within the protective section 105, air flow and circulation is possible 360 degrees in all directions without physically contacting any portion of the protective section or applying any pressure to the amputated wound site.

Protective section 105 includes an inverted U-shape truncated dome, cage or protective guard 115 to prevent blunt trauma to the stump and direct physical impact with another object by transferring any forces associated therewith away from the stump to the upper leg (e.g., the thigh). Cage or protective guard 115 is preferably designed and manufactured from materials taking into consideration several competing design factors. One design consideration already mentioned is that the cage or protective guard prohibit blunt trauma to the stump and direct physical impact with another object. Another significant design factor or consideration is that the cage or protective guard 115 maximizes air flow circulation 360 degrees in all directions about the stump. This later design factor is achieved by suspending the end of the stump within the cage or protective guard 115 without any physical contact therebetween. Since there is no physical contact between the stump and the protective guard, the interior surface of the protective section 105 is free of any fabric or cushioning. Total suspension of the end of the stump without any physical contact avoids pressure ulcers that would otherwise result from cutting off oxygen during restricted circulation. Protective section 105 therefore provides no physical support, whatsoever, to the lower leg limb. Furthermore, the material selected for the cage or protective guard 115 is sufficiently rigid to prevent blunt trauma to the stump and prohibit direct physical impact with an object while minimizing overall weight of the device. A polymer such as polyurethane may be used. Of course, some of these factors contradict each other and a balance therebetween has to be reached.

These design considerations are satisfied by designing the overall shape of the cage or protective guard 115 as an inverted U-shape truncated dome. Maximum air flow and circulation in all directions (360 degrees) about the stump is provided by configuring the protective guard 115 as an open (lattice work) structure with minimal obstructions and no moveable components. Here also competing factors must be balanced. On the one hand, preferably the open lattice structure has a minimum number of obstructions to maximize airflow. While on the other hand, the open lattice structure has to provide sufficient rigidity and protection to prohibit blunt trauma to the stump and direct physical contact with another object. In the preferred embodiment depicted in FIG. 1, within any single plane, preferably only a single intersecting or cross-member is employed to provide sufficient rigidity to the structure against impact while maximizing air flow and circulation. Of course, it is contemplated and within the scope of the present invention to use more cross-members sacrificing one of these two competing factors while enhancing the other.

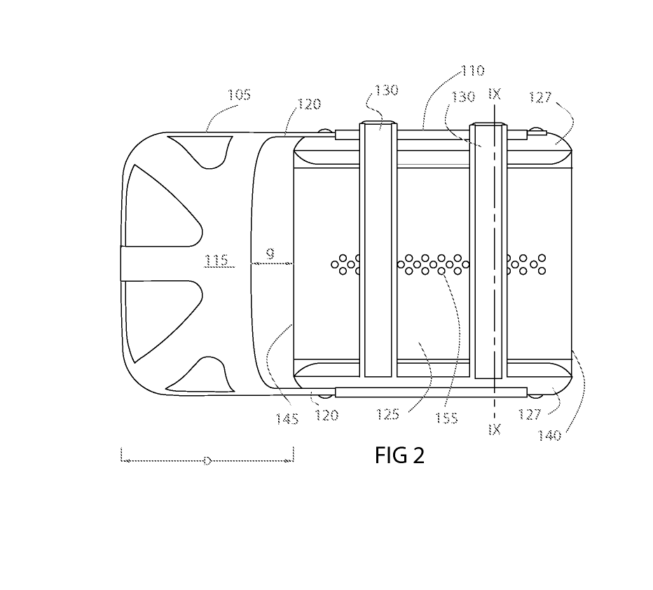

In the exemplary embodiment illustrated in the figures, cage or protective guard 115 does not entirely enclose the stump. Instead, only key areas of vulnerability of the stump from impact are targeted, namely the sides and end. When the patient is seated in a wheelchair the amputated leg is elevated substantially horizontal resting on a leg support with the end of the stump extremely vulnerable to impact with an object. Thus, cage or protective guard 115 is positioned in front of, along both sides and above the stump. Cage or protective guard 115 preferably does not extend beneath the stump. To maximize airflow and circulation in order to promote healing the end of the stump is totally exposed, i.e., free from any covering, bandage or wrapping. In this state the stump is not only susceptible to impact with another object, but also susceptible to catching or snagging on a sheet, blanket or other bed linen when drawn across the legs. To avoid such contact, the top of the cage of protective guard 115 is preferably domed or curved and extends only partially towards separated by a gap “g” from an interface with the supportive section 110, as shown in the top view in FIG. 2. The gap “g” as well as the openings in the lattice work structure itself allows the amputated wound site to be observed without having to remove the device. Sides 127 of the protective guard 115 are preferably substantially planar to conform to the substantially planar sides of the stump and are substantially parallel to one another.

Supportive section 110 includes a substantially U-shaped supportive bed, channel or well 125 with upward extending sides 127. A bottom surface 126 of the supportive bed, channel or well 125 is substantially planar to rest on the leg support of a wheelchair. One or more releasable and adjustable straps 130 extend laterally between the two sides 127 across the open top of the U-shaped supportive bed or well 125 for securing the patient's lower limb therein. Since the supportive section 110 is not full weight bearing (i.e., it does not support the entire weight of one's body while in a standing position), the adjustable straps 130 need not be tightly bound across the sides 127. Preferably, the adjustable straps 130 are loosely drawn across the sides 127 so as not to impede or restrict blood flow circulation to the stump thereby promoting healing.

Protective section 105 is releasably secured to the supportive section 110 via arms 120 extending from respective sides of the cage or protective guard 115 and received within complementary size and shape slots 150 defined in the sides 127 of the supportive section 110. Arms 120 are preferably substantially parallel to one another. When assembled; the front portion of the cage or protective guard 115 is separated from the end of the supportive bed 125 by a sufficient distance D (as shown in FIG. 2) to allow a portion of the stump to hang over the end of the supportive bed 125 unsupported and suspended in midair without physically contacting the protective guard 115. Preferably, the distance D is at least approximately 5 inches. By way of illustrative example, preferably approximately 2 inches-approximately 3½ inches, most preferably approximately 3 inches of the stump, hangs over the end of the supportive bed 125 so as to be unsupported and suspended in midair, while the distance separation between the end of the stump and the front of the protective guard 115 is at least approximately 2 inches. Once assembled together, the protective section 105 is stationary (non-pivoting) relative to the supportive section 110.

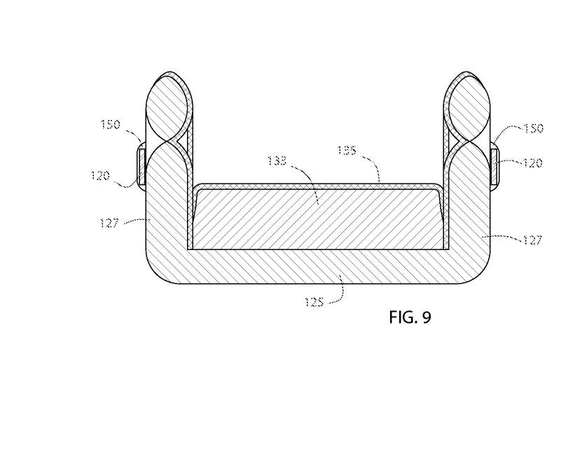

Referring to FIG. 9, disposed within the supportive bed, channel or well 125 is a core or pad layer 133 preferably made of foam. For sanitary reasons, the core layer is preferably covered by at least one outer fabric layer 135 that may be removed and laundered. Core layer 133 is preferably a relatively soft dual density breathable material (e.g., a polyurethane foam) that draws moisture away from the skin to help prevent further skin breakdown. Silver anti-microbial treated fabric 135 is preferred to kill harmful bacteria that may cause an unpleasant odor. An example of such fabric, is that commonly owned assignee of the present invention, under the name Air-O-Tech™, a three layer composite fabric including: (i) a bottom air and moisture ventilation layer; (ii) a spacer layer for permitting air flow; and (iii) a top soft and breathable anti-friction layer. The top anti-friction layer is subject to a nano silver treatment to impart anti-microbial/anti-bacterial/odor controlling properties.

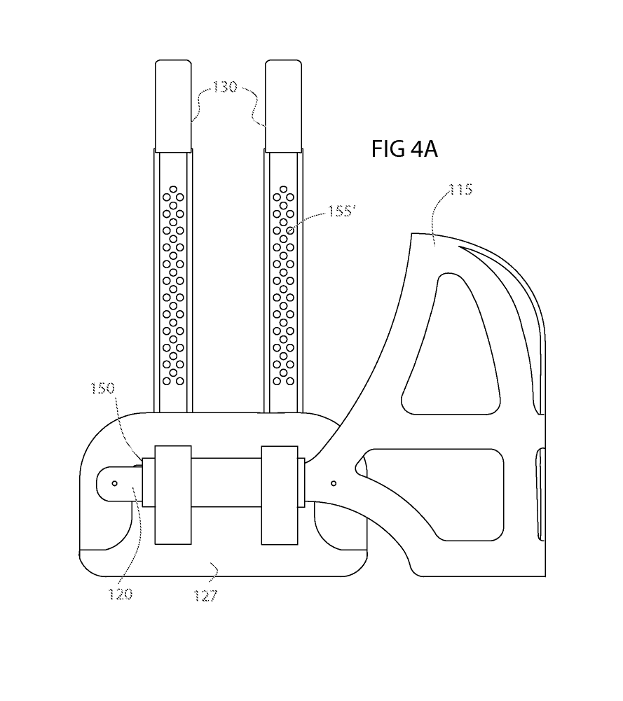

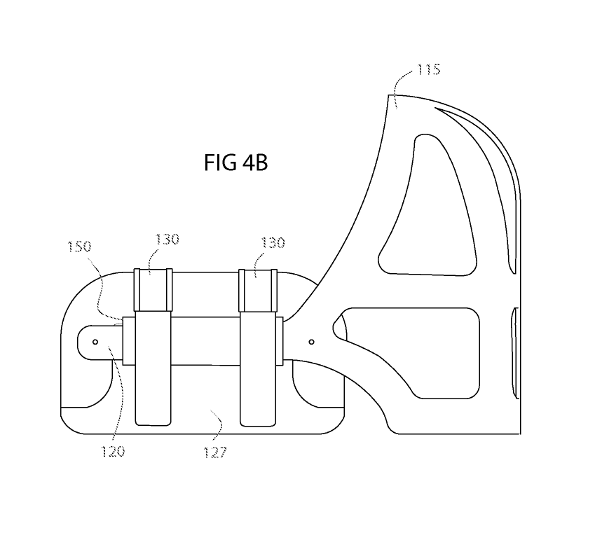

In a preferred embodiment, at least a portion of the outer fabric layer 135 includes a tactified region 155 to reduce the occurrence of leg slippage within the supportive bed 125. Any number of materials may be used for the tactified region, e.g., silicon or thermoplastic polyurethane (TPU). Tactified region 155 is depicted in FIG. 1 as a plurality of discrete sections grouped together so as to be substantially centered in a longitudinal direction. The tactified region may be modified, as desired, to include any number of one or more discrete sections the shape and size of which may be uniform or vary and the configuration of such sections may be modified in accordance with the present invention. Furthermore, the tactified region may be a single continuous shape, rather than a plurality of discrete sections. Similarly, a surface of the straps 130 in contact with the leg may also have a tactified region 155′ to reduce slippage, as depicted in FIG. 4A. Once again tactified region 155′ may be modified, as desired, and need not conform to that of tactified region 155.

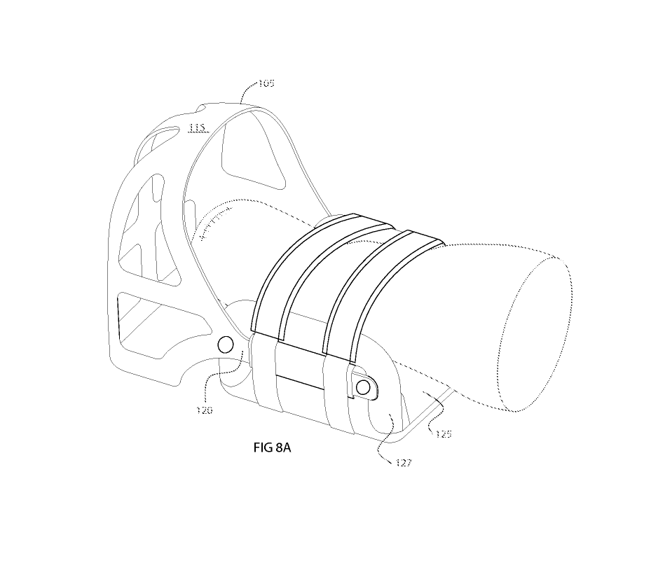

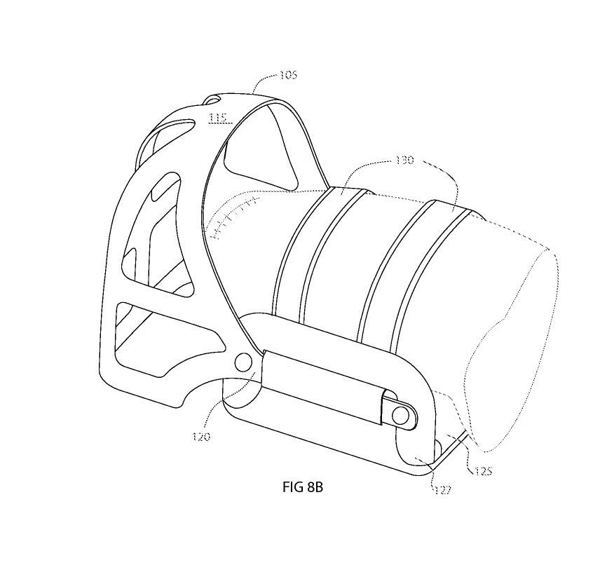

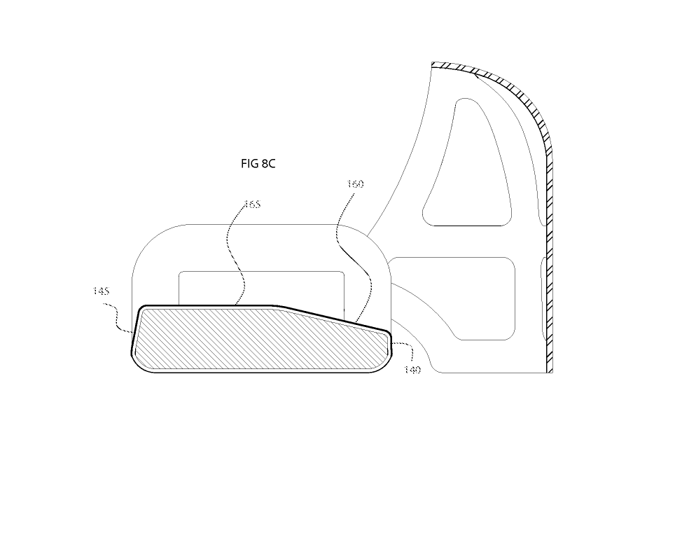

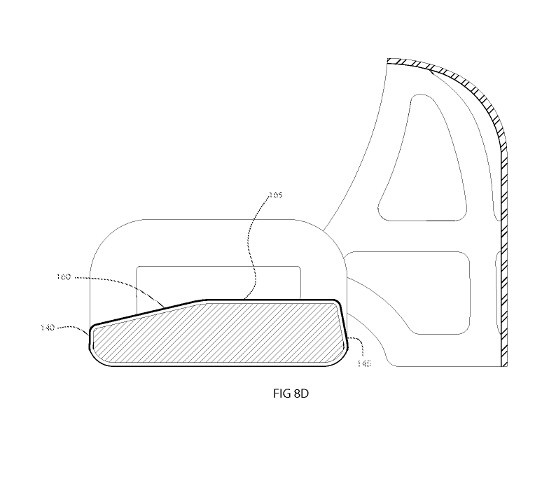

Referring to FIGS. 8A-8D, the present inventive protective and promotive healing device is universal in that it is ergonomically adapted to be used irrespective of whether the amputation is transtibial or transfemoral with the unique asymmetric shape of the core layer in a longitudinal direction and interchangeability of the end of the supportive section to which the protective section is secured while still maintaining the leg substantially horizontal. In particular, core layer 133 is symmetric along a lateral direction (as shown in FIG. 9), but asymmetric along a longitudinal direction (as shown in the cross-sectional view in FIGS. 8C & 8D). Referring once again to FIG. 9, in the lateral direction, the core layer 133 may be substantially planar or ergonomically curved to substantially conform to the back of the thigh. In a longitudinal direction, a first portion 165 of the top surface of the core layer 133 is substantially parallel with the bottom surface. However, as illustrated by the longitudinal cross-sectional view in FIGS. 8C & 8D, a second portion 160 of the core layer 133 slopes downward at an angle like a wedge to maintain the leg substantially horizontal when resting in the supportive section 110 which itself is horizontal. In a preferred embodiment, the angle at which the second portion 160 slopes relative to the first portion 165 is approximately 20 degrees. This angle or slope of the core layer in the longitudinal direction allows the supportive section 110 to be ergonomically adapted to conform to the calf, thigh or knee of the stump irrespective of whether the amputation is transtibial or transfemoral merely by selecting which side of the supportive section 110 to secure the protective section 105. In the case of a transtibial amputation, the protective section 105 is secured to a second end 140 of the supportive section 110, as illustrated in FIGS. 8A & 8C so that the tapered wedge substantially conforms ergonomically to the calf. Arranging the core layer 133 in such manner reduces pressure areas or points that may cause pressure ulcers. Whereas, if the amputation is transfemoral, the protective section 105 is secured to an opposite first end 145 of the supportive section (as illustrated in FIGS. 8B & 8D). The downward slope of core layer 133 toward the torso maintains the stump substantially level. Thus, irrespective of the type of amputation, core layer 133 substantially conforms to the amputated lower leg limb simply by rotating the supportive section 110 relative to the protective section 105 by 180 degrees.

During use, one of the two ends of the supportive section 110 is selected to be oriented to receive the arms 120 of the protective section 105 depending on the type of amputation (e.g., transtibial or transfemoral). Protective section 105 and supportive section 110 are releasably secured together by sliding arms 120 into respective recesses 150. Once assembled, the lower leg limb is placed in the supportive section 110 so that at least a portion of the stump hangs off the end of the supportive bed 125 suspended in midair without physically contacting the device. Preferably; approximately 3 inches of the stump hangs off the end of the supportive bed 125. Suspended in midair without physically contacting either the protective section 105 or supportive section 110, the stump is exposed to maximum air flow and circulation in all directions yet protected from blunt trauma by the protective guard 115. The preferred distance separation between the front of the protective guard 115 and the end of the stump is approximately 2 inches. Adjustable straps 130 are secured across the open top of the supportive bed 125 between the two sides 127 without exerting any pressure or impairing blood circulation to the leg and more particularly the stump. Supportive section does not impede, restrict or reduce blood circulation in the amputated lower leg limb.

While seated in a wheelchair with the amputated leg extending outward supported by the leg support, upon impact with another object, protective section 105 transfers, shifts or off-loads the forces associated therewith away from the stump to the upper portion of the leg (e.g., the thigh), thereby preventing blunt trauma to and direct physical contact with the stump. Moreover, the curved protective guard 115 allows a blanket, sheet, bed linen or any other fabric to pass easily over the stump preventing any physical contact therewith. Moreover, since the end of the stump is suspended within the protective guard 115 without physically contacting either, the protective section 105 or the supportive section 110 optimum air flow and circulation is provided to promote healing of the amputated site. A single device may universally be used with either a transtibial or transfemoral amputation by merely rotating the supportive section 110 relative to the protective section 105 by 180 degrees and securing the arms 120 in the respective slots 150. Accordingly, the same device may interchangeably be used on either the right or left side of the body. The present inventive device is not a limb replacement and thus is non-weight bearing in that it is not adapted to bear or support the full weight of the person while in a standing position. Instead, the device in accordance with the present invention provides protection to and promotes healing of the amputated wound site.

Thus, while there have been shown, described, and pointed out fundamental novel features of the invention as applied to a preferred embodiment thereof, it will be understood that various omissions, substitutions, and changes in the form and details of the devices illustrated, and in their operation, may be made by those skilled in the art without departing from the spirit and scope of the invention. For example, it is expressly intended that all combinations of those elements and/or steps that perform substantially the same function, in substantially the same way, to achieve the same results be within the scope of the invention. Substitutions of elements from one described embodiment to another are also fully intended and contemplated. It is also to be understood that the drawings are not necessarily drawn to scale, but that they are merely conceptual in nature. It is the intention, therefore, to be limited only as indicated by the scope of the claims appended hereto.

Every issued patent, pending patent application, publication, journal article, book or any other reference cited herein is each incorporated by reference in their entirety.

1. A post operative amputated lower leg limb protective and promotive healing device comprising:
a supportive section having a first end and an opposite second end; and
an open framework protective section releasably securable to at least one end of the supportive section; the open framework protective section forming a plurality of openings to allow airflow therethrough in all directions; and the open framework protective section being configured to insure no physical contact by the protective section with the amputated lower leg limb; wherein the open framework protective section is non-weight bearing.

2. The device in accordance with claim 1, wherein the open framework protection section has no movable components.

3. The device in accordance with claim 2, wherein the open framework protective section is non-pivoting with respect to the supportive section.

4. The device in accordance with claim 1, wherein the open framework protective section is configured so that it provides no support whatsoever for the amputated lower leg limb.

5. The device in accordance with claim 1, wherein the supportive section does not impede blood circulation in the amputated lower leg limb.

6. The device in accordance with claim 1, wherein the protective section has an interior free from any fabric.

7. The device in accordance with claim 1, wherein the protective section further comprises a pair of substantially parallel extending arms; the protective section being interchangeably and releasably securable to the first or second ends of the supportive section via the pair of substantially parallel extending arms receivable within complementary slots formed in the supportive section.

8. The device in accordance with claim 1, wherein the supportive section comprises in a lateral direction a cross-sectional substantially U-shaped supportive platform having an open top.

9. The device in accordance with claim 8, wherein the supportive section comprises at least one adjustable and releasable securing mechanism extending laterally across the open top of the supportive platform.

10. The device in accordance with claim 9, wherein the at least one adjustable and releasable securing mechanism is a strap with a contacting surface at least a portion of which is covered with a tactified region to provide traction and reduce slippage.

11. The device in accordance with claim 1, wherein the supportive section comprises a supportive platform forms a longitudinal recess in which an ergonomic wedge shaped core layer is disposed therein.

12. The device in accordance with claim 11, wherein the core layer is ergonomically adapted in a longitudinal direction for use with a transtibial lower leg limb amputation or transfemoral lower leg limb amputation depending on whether the protective section is assembled to the first or the second end of the supportive section.

13. The device in accordance with claim 11, wherein the core layer is symmetric in a lateral direction, but asymmetric in a longitudinal direction.

14. The device in accordance with claim 13, wherein the core layer is curved in the lateral direction and a portion of the core layer slopes downward in a longitudinal direction from one end towards an opposite end.

15. The device in accordance with claim 11, wherein the core layer is a dual density breathable material that draws in moisture.

16. The device in accordance with claim 11, wherein the supportive section further comprises an outer layer covering at least a portion of the core layer, wherein the outer layer is a three layer composite fabric including: (i) an air and moisture ventilation bottom layer; (ii) a spacer layer permitting air flow; and (iii) a breathable anti-friction top layer subject to a nano silver treatment to impart anti-microbial/anti-bacterial/odor controlling properties.

17. The device in accordance with claim 16, wherein the outer layer as a tactified region to provide traction and reduce slippage.

18. A method for using the post operative amputated lower leg limb protective and promotive healing device in accordance with claim 1, comprising the step of:
placing the amputated lower leg limb into the supportive section until at least a portion of an end of the amputated lower leg limb extends beyond the second end of the supportive section secured to the protective section, that portion of the amputated lower leg limb extending beyond the second end of the supportive section remaining free from any support or physical contact whatsoever with the device.

19. The method in accordance with claim 18, wherein the end of the amputated lower leg limb is disposed with sufficient clearance with respect to the protective section to offload energy away from the end of the amputated lower leg limb upon impact of the protective section with an object.

20. The method in accordance with claim 18, wherein the end of the amputated lower leg limb is separated from the protective section by a gap of at least two inches; and at least three inches of the amputated lower leg limb extends beyond the end of the supportive section.

21. The method in accordance with claim 18, prior to the inserting step, further comprising the steps of:
selecting either the first or second end of the supportive section on which to secure the open framework protective section depending on whether the amputated lower leg limb is transtibial or transfemoral; and
assembling the open framework protective section to the selected first or second end of the supportive section.

22. The method in accordance with claim 21, wherein the supportive section comprises in a lateral direction a cross-sectional substantially U-shaped supportive platform having an open top.
